(19) United States
(12) Patent Application Publication
(10) Pub. No.: US 2011/0033595 A1
(43) Pub. Date: Feb. 10, 2011

(54) Fatty acid fractionation process, fatty acid products and use thereof

(76) Inventors: Rudolf Krumbholz, Merchweiler (DE); Peter Lembke, Gersheim (DE)

Correspondence Address: FERRELLS, PLLC, P. O. BOX 312, CLIFTON, VA, 20124-1706, US

(21) Appl. No.: 12/462,840

(22) Filed: Aug. 10, 2009

(51) Int. Cl. C11B 5/00; C11B 7/00; A61K 31/20; A61P 43/00

(52) U.S. Cl. 426/546; 554/175; 554/184; 554/212; 554/189; 514/560

(57) ABSTRACT

The present invention relates to a concentration- and purification method for compositions containing phytanic acid and/or derivatives thereof besides other fatty acids and/or their derivatives, preferably besides polyunsaturated fatty acids and/or their derivatives, such as esters or salts. With the fractionation process the phytanic acid (derivative) can become concentrated and the fatty acid mixture, preferably PUFA-mixture, can be purified from this particular component.

### BACKGROUND OF THE INVENTION

As active ingredient in pharmaceutical drugs and topical formulations phytanic acid (hereinafter “PhA”) is of importance and there is a need for highly concentrated product from natural sources. On the other side, PhA may represent an important health risk if elevated quantities are taken on a regular basis over a long period of time. One of the richest natural sources of PhA is fish oil, algae oil and in general marine oils. These sources can contain up to 1.000 ppm and even more of PhA compared to normal levels found in other foods of less than 10 ppm. On the other side, marine oils are also the most important natural source of omega-3 fatty acids. There is a worldwide understanding that especially the western diet has a far too low content in omega-3 fatty acids and therefore every country has established recommended daily Intakes of EPA+DHA that vary from 200 mg per day up to 1.000 mg per day. In order to satisfy these recommendations more and more fish oil and omega-3 concentrates are consumed on a regular daily basis. This has turned the fish oil/omega-3 market in one of the strongest growing markets in the world. However, together with the increased fish oil consumption, which is one of the most important natural PhA sources found on earth, a simultaneously increased and uncontrolled PhA up-take is noted. This could lead to serious health problems of millions of consumers.

For a long time this PhA content in food was only seen as a problem for a very small group of people suffering from insufficient alpha-oxidation in the liver. This in turn resulting in a continuous increase of the PhA concentration in the blood serum of these subjects, where it competes directly with the DHA in the sn-2 position of the phospholipids and is the cause of numerous severe health risks for the individual.

The corresponding disorder is known as “Heredopathia Atactica” or better known as “Refsums Disease”. Later it was discovered that PhA plays a vital in various human pathologies like Refsums syndrome, Zellweger syndrome, neonatal adrenoleuko-dystrophy, rhizomelic punctata chondrodysplasia and others more.

Latest research studies showed that the potential toxic risks triggered already by very small PhA concentrations are far more amplified as originally believed. Today it is known that if PhA is consumed on a regular basis in food supplements or food compositions, in elevated concentrations, there are several risks associated to it:
1. PhA may induce several types of cancers like cancer of the lungs, breast, prostata and colon.
2. PhA is cytotoxic, especially for the mitochondria. It is one of the strongest cause for programmed cell death (apotosis). Note: Omega-3 fatty acids, especially EPA and DHA are known for their apotosis inhibitioning effect.
3. PhA is strongly pro inflammatory because it displaces the EPA and/or DHA from the sn-2 position of the phospholipids found in the cell membrane.
4. PhA is in many aspects a direct antagonist to EPA and DHA.

Taking all these threats for human health coming from PhA contamination in marine oils into account, it is clear that there is an urgent need for a controlled PhA-free or at least strongly PhA-reduced omega-3 or omega-6 oil no matter from what source it may originate.

On the other hand, PhA may be used in topical applications to improve the blood circulation and/or in drug formulations for treatment of selected diseases, like for example if there is a need of a certain controlled angiogenesis or for the treatment of human type 2 diabetis and obesity (SCHLÜTER, YUBERO, et al. in Internat. Journ. Of Obesity (2002), 26, 1277-1280). Thus, for pharmaceutical purposes it is necessary to have a concentrated product which has a defined content of PhA.

Polyunsaturated fatty acids (hereinafter “PUFA” or “PUFA's”), especially long chain omega-3 fatty acids, such as stearidonic acid (STD), eicosatetraenoic acid (ETA), eicosapentaenoic acid (EPA), docosapentaenoic acid (DPA), docosahexaenoic acid (DHA) or their esters, such as their ethyl esters, have certainly become one of the most popular food supplements during the last couple of years. Most long-chain omega-3 or omega-6 fatty acids (such as C₂₀-C₂₂) come from fish oil. The annual world-wide fish oil production is estimated at around 600,000 to 1 million metric tons. However, only about 3% of this fish is intended for human consumption.

Besides omega-3 fatty acids fish oils contain omega-6 fatty acids and omega-9 fatty acids or derivatives thereof. Examples for omega-6 fatty acids are gamma-linolenic acid (GLA), arachidonic acid (AA). An example for omega-9 fatty acids is oleic acid.

Apart from the classical fish oil as source of omega-3, omega-6 and omega-9 fatty acids, algae oils, bacterial oils, fungus oils and specialty oils, such as squid and calamari oils, mussels oils or krill oils, are becoming more and more popular.

Generally the best source of highly unsaturated PUFA's comes from marine cold water environments. Typical representatives for highly unsaturated PUFA's are EPA and DHA, next to DPA, ETA and SDA. The most characteristic methylated (“branched”) fatty acids found in many marine oils are the phytanic acid and it's oxidized form, the pristanic acid (hereinafter “PA”). They originate from the phytol alcohol rest of the chlorophyll. Hence, especially high amounts of PhA are found in the oil from algae and algae's consuming species, such as small fish, shrimp, krill, mussels or squid. While the majority of food contains less than 10 ppm PhA, fish oils and other marine oils mentioned above can easily reach 750 to 1000 ppm of PhA and even more.

### PRIOR ART

PhA is a known additive for the cosmetic industry or in drug formulations. US 2008/00 76 721 A1 describes use of amino acid salts comprising naturally occurring methylated branched chain fattys acids, inter alia phytanic acid or pristanic acid, for cosmetic, dietary supplement or pharmaceutical compositions which are useful in increasing the variables associated with physical performance for the regulation of athletic function.

WO 2005/000036 A1 discloses livestock products with an increased PPAR/RXR heterodimer activator level, the PPAR/RXR heterodimer activator being phytanic acid, or a metabolite or derivative thereof.

WO 2006/114140 A1 discloses pristanic acid-containg cosmetic preparations.

WO 2004/017766 A1 discloses nutraceutical compositions comprising biotin and at least one additional ingredient, for example phytanic acid.

It is known that PhA is mainly attached to triglycerides, phospholipids and/or cholesterol esters present in an oil. Therefore, it cannot be removed efficiently by the classical refining steps which every marine oil undergoes. Experiments demonstrate that molecular distillation of the triglycerides (TG's) in order to remove the pesticides and other contaminants is not very effective.

Normal distillation and/or molecular distillation of the ester's or FFA's is also not an effective solution because the boiling point of PhA is similar to the one of the long chain poly unsaturated fatty acids (PUFA's).

Even the classical winterization/crystallization technology is not applicable because the branched PhA crystallizes at very low temperatures together with the EPA and DHA.

Consequently the PhA is concentrated alongside with the PUFA's during all these processes, explaining why especially highly concentrated EPA and DHA formu-lations contain the highest PhA concentrations.

In other words, all these so far discussed classical purification and concentration technologies generally applied in the oil industry indeed lead to higher concentrated EPA and DHA products but at the same time to a non-desired higher concentrated PhA content in that particular product (food, supplement or drug).

Several purification methods for PUFA's have been disclosed in the patent literature.

WO 01/10809 A1 discloses a recovery of polyunsaturated fatty acids from urea adducts by subjecting said products to an extraction treatment using subcritical or supercritical fluids at a temperature of not above 70° C.

EP 1 157 692 A1 discloses a composition of fatty acids containing at least 80% by weight of EPA and DHA or their derivatives. These compositions are obtained from fish oil by subjecting said oil to different refinement treatments. The reported purification process encompasses transesterification followed by urea fractionation and by distillation processes.

From U.S. Pat. No. 5,656,667 a fatty acid composition is known comprising at least 80% by weight of a combination of EPA and DHA and comprising in addition at least 1% by weight of (all Z omega-3)-6,9,12,15,18-heneicosapentaenoic acid. Another fatty acid composition referred to in this patent is a mixed fatty acid composition comprising at least 80% by weight of omega-3 fatty acids, at least 80% by weight of the total fatty acid content being a combination of EPA and DHA in an amount from 1:2 to 2:1 and at least 1.5% by weight of the total acids being omega-3 fatty acids other than EPA and DHA. These compositions are obtained from fish oil by subjecting said oil to different refinement treatments. The reported purification process encompasses transesterification followed by urea fractionation and by molecular distillation process.

Purification of PUFA by chromatographic methods or by extraction methods has also been already disclosed in the prior art. In WO 00/71650 A1 a method for purifying natural oils is described. In this method a natural oil comprising unsaturated higher fatty acids is purified by treating said natural oil with alumina as a sorbent. This treatment results in tasteless and odourless PUFA compositions. WO-A-01/07137 A1 discloses a column chromatography separation method for compositions of matter. Therein a fluid in liquid, non-subcritical and non-critical state is used as an eluent. This method can be used, for example, for the purification of natural PUFA-containing oils.

### SUMMARY OF INVENTION

As discussed above it is impossible to separate the PhA from the fatty acid mixtures by means of the classical oil refining methods.

Surprisingly it has been found, that PhA can be separated from natural fatty acid mixtures, preferably marine oil, by passing said fatty acid mixture through a chromatographic column and using a liquid or supercritical eluent.

Thus the objective of the present invention is the provision of a process which is easy to implement and which separates PhA and/or its derivatives from fatty acid mixtures. As a consequence of this process a composition comprising a fatty acid mixture, preferably polyunsaturated fatty acids and/or their derivatives is obtained with considerably increased efficacy and reduced health risk for the consumer of said composition due to their low or not existing PhA concentration. Furthermore, fatty acid fractions are obtained comprising increased amounts of PhA and/or its derivatives.

Still further features and advantages of the invention are apparent from the following description.

### BRIEF DESCRIPTION OF DRAWINGS

The invention is described in detail below with reference to the appended drawings. In the Figures:

FIG. 1 is a section of a gas chromatography (GC) chromatogram of fractions of a PhA-containing fish oil fractionated according to the invention as disclosed in Example 1;

FIG. 2 is a GC chromatogram of a PhA-enriched fraction derived by fractionation according to the invention of fractions one and two from Example 1;

FIG. 3a is a GC chromatogram profile of a commercially available EPA/DHA formulation;

FIG. 3b is an example given in the European Pharmacopoeia 6.3 Monograph Omega-3 Acid Ethyl Esters 90; and

FIG. 3c is a GC chromatogram profile of a fractionation product according to the invention containing less than 50 ppm PhA.

### DETAILED DESCRIPTION

The invention is described in detail below with reference to several embodiments and numerous examples. Such discussion is for purposes of illustration only. Modifications to particular examples within the spirit and scope of the present invention, set forth in the appended claims, will be readily apparent to one of skill in the art. Terminology used herein is given its ordinary meaning consistent with the exemplary definitions set forth immediately below.

The present invention relates to process for fractionation of chromatographic feed material comprising fatty acids and/or derivatives thereof including phytanic acid and/or its derivatives said process comprises subjecting said chromatographic feed material to column chromatography using a liquid or a supercritical fluid as an eluent and collecting a plurality of eluate fractions which contain increased amounts of phytanic acid and/or of its derivatives and which contain reduced amounts of phytanic acid and/or of its derivatives.

The term “fatty acid” is to be understood in this specification as a long chain carboxylic acid having at least six carbon atoms. Fatty acids may be saturated or unsaturated compounds. These compounds may contain linear or branched alkyl groups, for example linear or branched alkyl groups having between six and thirty carbon atoms. Preferably fatty acids comprise one or very preferred more than one, for example two, three or four ethylenically unsaturated carbons in the alkyl chain. Very preferred mixtures of fatty acids comprise PUFA's.

The term “fatty acid derivative” is to be understood in this specification as any compound comprising one or more groups derived from fatty acids. Examples of fatty acid derivatives are salts of fatty acids, for example alkali salts, esters of fatty acids, preferably fatty acid ester group containing lipids, such as fatty acid ester group containing glycerolipids, fatty acid ester group containing glycerophospho-lipids, fatty acid ester group containing sphingolipids, fatty acid ester group containing sterol lipids, fatty acid ester group containing prenol lipids, fatty acid ester group containing saccharolipids and fatty acid ester group containing polyketides. Further examples for fatty acid derivatives are amides of fatty acids.

Preferred fatty acid derivatives are fatty acid esters with polyhydric alcohols, preferably with glycerol. The different hydroxy groups of polyhydric alcohols, such as of glycerol, are preferably esterified with different fatty acid groups.

Phytanic acid (PhA) is a specific fatty acid. Derivatives of PhA are as defined above for fatty acids. Preferred derivatives of PhA are salts of PhA and/or are esters of PhA.

In the process of this invention a chromatographic feed material is used which contains phytanic acid and/or its derivatives and in addition further fatty acids and/or their derivatives.

Typical concentrations of the combined content of PhA and its derivatives in chromatographic feed materials are 100-1500 ppm or higher.

During the process fractions with high contents of PhA and/or its derivatives and with low contents of PhA, PA and/or their derivatives are obtained.

The term “low content” is to be understood in this specification as 100 ppm or lower, preferably between 0 and 100 ppm, very preferably between 0 and 50 ppm.

The term “high content” is to be understood in this specification as higher than 100 ppm, preferably higher than 300 ppm, and very preferred between 3000 ppm and 100%.

The fractions obtained in the process of this invention can contain-depending on the feed material PhA only, PhA derivatives only, or any combination of two or more of these compounds or compound classes.

In the process of this invention any substrate comprising PhA and/or their derivatives and fatty acid mixtures, preferably including polyunsaturated fatty acids and/or their derivatives can be used as a feed material. Preferably the substrate is used in form of an oil.

PhA can be present in the form of the free acids, their salts or their derivatives, such as esters or amides. Any combination of PhA and/or their derivatives can be present in the substrate to be used in the process of this invention. Preferably, the substrate contains PhA in the form of the free acid or as an alkyl ester, such as an ethyl ester.

Fatty acid mixtures, preferably PUFA's containing PhA can also be present in the form of the free acids, their salts or preferably as derivatives thereof, such as esters or amides, very preferably as alkyl esters, especially as ethyl esters. Any combination of fatty acid mixtures in the form of free acids, salts or derivatives can be present in the substrate to be used in the process of this invention.

As a substrate for column chromatography a PhA-containing oil can be used directly.

Preferably the triglycerides in naturally occurring oils are trans-esterified to the corresponding fatty acid alkyl esters, very preferably to the corresponding ethyl esters. This treatment also results in a formation of alkyl esters of PhA.

Thus, a preferred process of this invention comprises the steps of:
a) subjecting an oil comprising more than 100 ppm of phytanic acid and/or of derivatives thereof and comprising triglycerides containing polyunsaturated fatty acid groups to a transesterification to result in a composition comprising alkyl esters of polyunsaturated fatty acids,
b) optional but preferred: subjecting the alkylester composition to an urea fractionation process where the saturated and monounsaturated fatty acids and their derivates are eliminated to a large extent.
c) subjecting the composition comprising alkyl esters of polyunsaturated fatty acids to column chromatography using a liquid or a supercritical fluid as an eluent,
d) collecting separate fractions from the treatment of step c), and
e) continuing processing of those fractions having a content of phytanic acid and/or their derivatives of above 100 ppm to result in a product comprising fatty acids, salts thereof and/or derivatives thereof and having a total content of phytanic acid and/or of its derivatives of at least the triple concentration level of PhA and/or of its derivatives as the initial feed.

In an alternative embodiment, a preferred process of this invention comprises the steps of:
a) subjecting an oil comprising more than 100 ppm of phytanic acid and/or of derivatives thereof and comprising triglycerides containing polyunsaturated fatty acid groups to a transesterification to result in a composition comprising alkyl esters of saturated-, monounsaturated- and polyunsaturated fatty acids, next to the PhA and/or its derivatives,
b) subjecting the composition comprising alkyl esters of polyunsaturated fatty acids to column chromatography using a liquid or a supercritical fluid as an eluent,
c) collecting separate fractions from the treatment of step b), and
d) continuing processing of those fractions having a content of phytanic acid and/or of its derivatives of below 100 ppm, preferably of below 50 ppm to result in a product comprising polyunsaturated fatty acids and/or derivatives thereof characterized by a total content of phytanic acid and/or of its derivatives of below 100 ppm, preferably of below 50 ppm.

Continued processing of the fractions resulting from steps a) to c) can encompass subjecting these fractions to another column chromatography process, combining two or more fractions to obtain a fatty acid (derivative) composition with a desired spectrum of fatty acid (derivatives), modifying the fatty acid (derivatives) in one or more fractions, for example by deesterification, transesterification, salt formation or other chemical treatment or a combination of two or more of these continued processing steps.

By a selective combination of different chromatographic column packing materials and/or by further processing according to this invention, any desired PhA concentration can be obtained.

In a preferred process of this invention an oil is used as a substrate in step a) which contains phytanic acid as well as triglycerides containing polyunsaturated fatty acid groups.

In the process of this invention any fluid in a liquid or supercritical state can be used as an eluent. Typical examples for an eluent are the following fluids in a liquid or supercritical state: dinitrogen oxide, fluorohydrocarbons, fluorochlorohydro-carbons, carbon dioxide, sulfur hexafluoride, hydrocarbons, preferably propene, propane or ethane, ammonia, sulfur dioxide, xenon, heptane, hexane, pentane or a mixture of two or more thereof.

A very preferred eluent is liquid or supercritical carbon dioxide (CO₂).

The fluid used as an eluent can be used as such. Optionally the liquid or super-critical fluid contains a polar modifier, preferably methanol, ethanol, isopropanol or water.

The PhA (derivative) is eluated as a separate fraction or as a fraction in combination with fatty acids. Preferably the PhA (derivative) is eluated as a selected fraction.

The isolation and/or removal of the PhA (derivative) from a fatty acid mixture, preferably polyunsaturated fatty acids can be performed at any stage of the purification chain where column chromatography is used.

In the processes of this invention one or more chromatographic columns can be used, which is/are packed with a particulate or different organic and/or inorganic materials. This material can be used in unmodified form or it can be functionalized by covalent binding with groups which modify the surface of said material. Preferred surface modifying groups are aminopropyl groups, cyanopropyl groups, diol groups, phenyl groups or hydrocarbon groups. These latter groups modify a particulate and polar organic or polar inorganic material into a reverse phase material. Preferably such reverse-phase material is modified with RP-8 or with RP-18.

In very preferred processes of this invention the chromatographic column is packed with silica, in particular with silica modified with aminopropyl, with cyanopropyl, with diol, phenyl or with hydrocarbons, preferably with reverse-phase RP-8 or with reverse-phase RP-18.

In other very preferred processes of this invention the chromatography column is packed with aluminium oxide, in particular with aluminium oxide modified with aminopropyl, with cyanopropyl, with diol, phenyl or with hydrocarbons, preferably with reverse-phase RP-8 or with reverse-phase RP-18.

Different sources can be used for the production of oils which are treated and/or used as a PhA-source according to the invention. One preferred source of oil is an oil of plant origin.

Another preferred source of oil is an oil of marine origin, preferably a fish oil, a calamari oil, a squid oil, a krill oil, a marine algae oil, a mussel oil or a clam oil.

Another preferred source of oil is an oil which is produced by microbes, preferably by algaes, yeasts or bacteria, in heterotrophic or autotrophic industrial fermenters, open tubes, flat panel reactors, open pond systems or other systems.

In a preferred variant of the process of this invention the continuing processing comprises a combination of fractions from the column chromatography to result in a product having a content of EPA and DHA alkyl esters of at least 60% by weight and containing no or up to 100 ppm, preferably below 50 ppm of phytanic acid and/or of their esters.

In a very preferred variant of the process of this invention the continuing processing comprises a combination of fractions from the column chromatography to result in a product having a content of EPA and DHA alkyl esters of at least 80% by weight and containing no or up to 100 ppm, preferably below 50 ppm of phytanic acid and/or of their esters.

In other process variants those fractions of the column chromatography are used which result in a product having a content of EPA and DHA alkyl esters of at least 80% by weight and wherein the weight proportion of EPA and DHA alkyl ester in said product is between 4:1 and 1:5 and very preferred between 2:1 and 1:2.

In still other process variants those fractions of the column chromatography are used which result in a product having a content of EPA and DHA alkyl esters of at least 85% by weight and having in addition a content of other omega-3 C₂₀-C₂₂-fatty acid alkyl esters below 3% by weight. Said other omega-3 C₂₀-C₂₂-fatty acid alkyl esters are preferably selected from the group of alkyl esters of DPA, ETA and 21:5.

In still other process variants those fractions of the column'chromatography are used which result in a product having a content of EPA and DHA alkyl esters of at least 85% by weight and having in addition a content of other omega-3 21:5-fatty acid alkyl esters below 1.5% by weight.

After the substrate has been purified by column chromatography and separated into different fractions the continuing processing can encompass different steps. In one embodiment, two or more fractions can be mixed to result in a PhA-free PUFA-composition of desired fatty acid spectrum. In another embodiment or in a subsequent treatment the alkyl esters can be modified to result in a fatty acid mixture and/or a fatty acid salt mixture and/or another fatty acid derivative mixture.

In a preferred process variant the continuing processing comprises a de-esterification of polyunsaturated fatty acid alkyl esters to result in polyunsaturated fatty acids and/or salts thereof.

These PhA free or reduced purified PUFA (derivatives) can be used as additives for food compositions, as nutrients and preferably as pharmaceutical compositions.

Still another embodiment of this invention relates to the use of a liquid or super-critical fluid as an eluent in a column chromatographic process to reduce the concentration of phytanic acid and/or of their esters in compositions containing fatty acid mixtures, preferably polyunsaturated fatty acids and/or polyunsaturated fatty acid esters.

The following examples illustrate the invention without limitation.

### Example 1

A commercially available fish oil (EPA content 20%, DHA content 9%) was transformed to the ethyl ester with ethanol and sodium ethylate as catalyst according the standard procedure. A large part of the saturated and mono unsaturated fatty acid ethyl esters were removed by standard state of the art urea precipitation. The resulting product contained 45% EPA, 19% DHA and 4000 ppm PhA. This product was used as starting material for further processing and was injected (2 kg) onto a 150 liter stainless steel fractionation column packed with a mixture of diol- and aminopropyl silica material at 52° C., 163 bar and a CO₂ flow of 700 kg/h. The eluate was collected in eight fractions (F1.1. to F.4.2.) The PhA was enriched especially in fraction one (F1.1.) and two (F1.2.) up to a content of more than 10%. Fraction three (F.2.1.) to fraction eight (F.4.2.) usually contain no, or less than 100 ppm PhA.

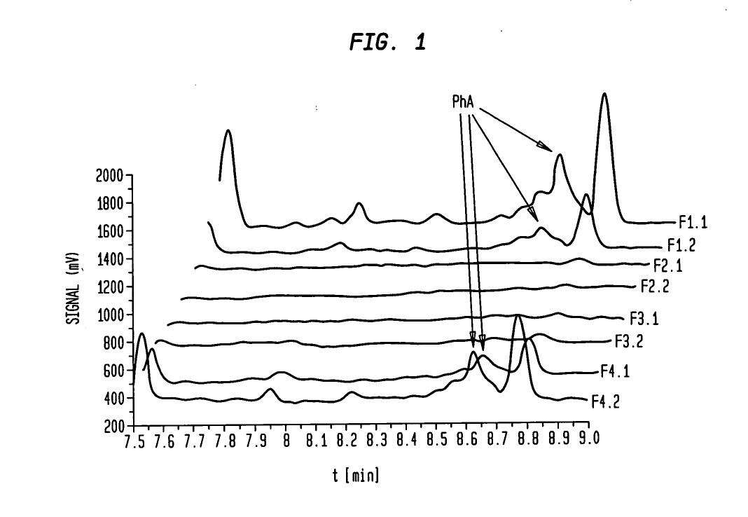

FIG. 1 shows the first section (between 7.5 and 9.0 min) of the GC chromatograms of the eight fractions collected, after injecting a EPA/DHA (45/19) fish oil containing 4000 ppm PhA onto the fractionation column. In this case the injections were run in the overlapping modus. This means that new material is injected batch wise onto the separation column before all fractions of the previous run have left the column. This modus has the consequence that the first fraction(s) of the second run co-elutes with the last fractions of the first run. This explains the reappearance of PhA in F4.1. and F4.2. shown in FIG. 1. This overlapping modus is used to produce highly concentrated EPA products (F.2.1. to F.3.2.) containing less than 100 ppm PhA.

Should the process be run in the standard mode (not overlapping mode), then the DHA fractions F4.1. and F4.2. are also free of PhA.

In FIG. 1 a section of the GC-profile of fractions F1.1. to F4.2. is shown which are obtained by the fractionation process (in the overlapping modus). In FIG. 1 the evolution of the PhA peak is demonstrated.

### Example 2

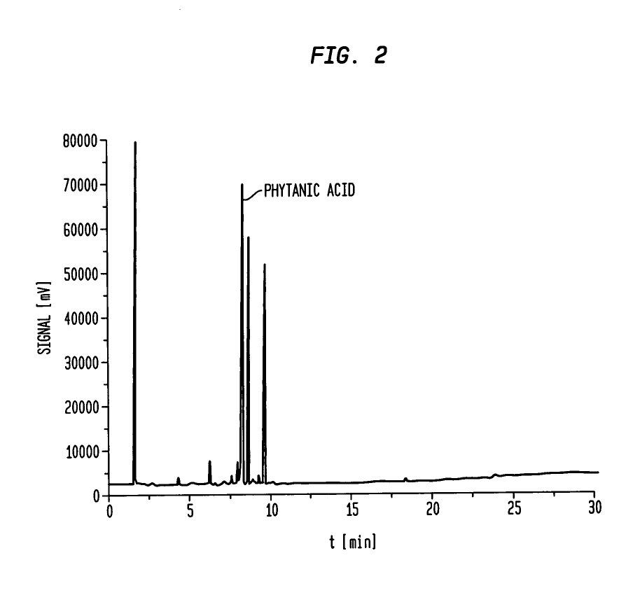

The collected fractions one and two from example one were used as starting material for further enrichment of PhA. This starting material contained 8% PhA. The reprocessing was done under the same conditions as under example one. Fraction one of the reprocessed product contained 41.07% PhA. This is shown in FIG. 2, t_{R} 8,067.

### Example 3

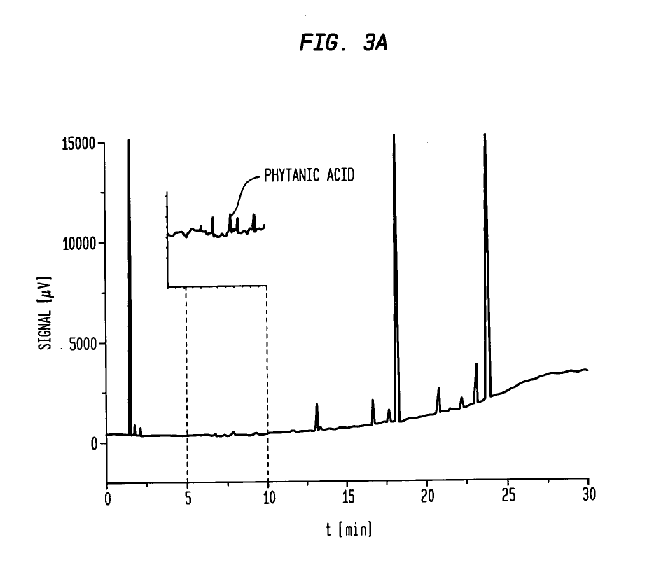

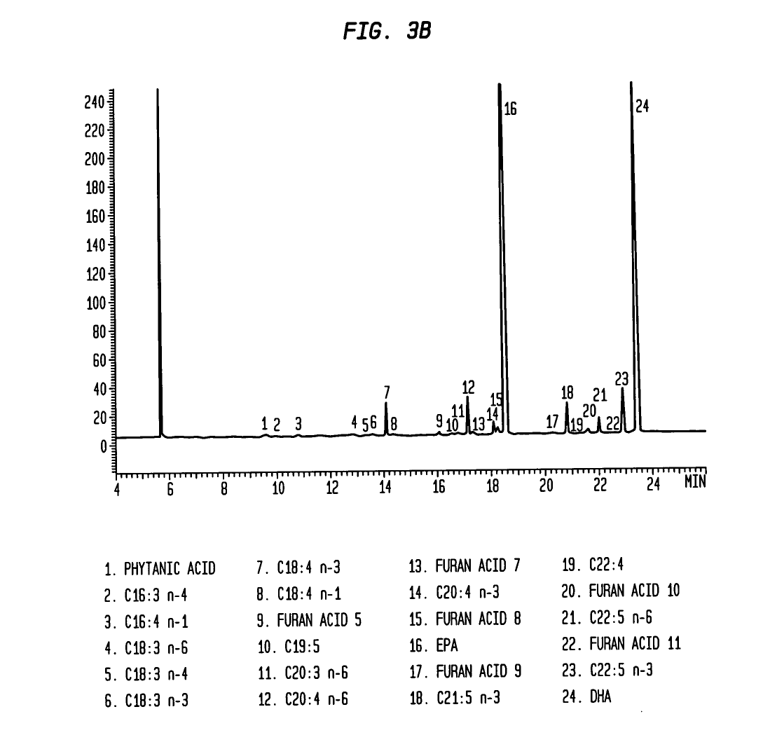

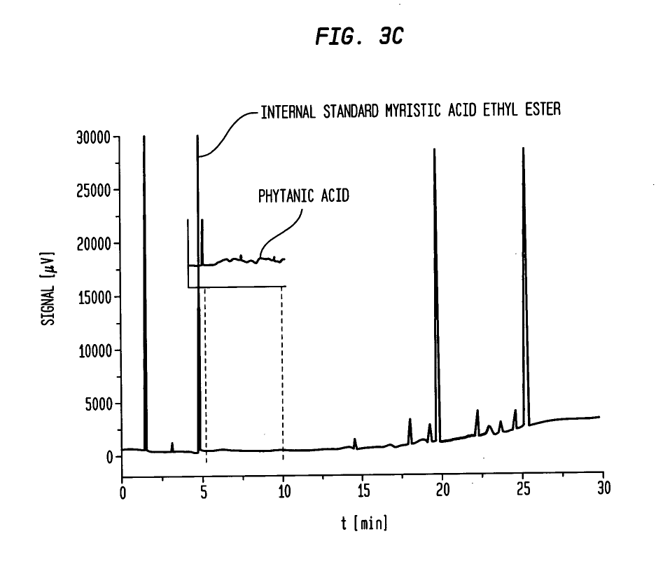

FIG. 3a shows the GC chromatogramm of commercial available EPA/DHA formulation containing 84% EPA+DHA and 1600 ppm PhA.

The chromatogram shown in FIG. 3a complies with the example given in the European Pharmacopoeia 6.3 Monograph Omega-3 Acid Ethyl Esters 90 which is attached as FIG. 3b.

For example 3, a feed similar to the product according FIG. 3a was used as a starting material for the supercritical fluid chromatography (“SFC”) process. 5 kg of this feed was injected onto the fractionation column under the same conditions as in example 1. Eight fractions were taken. Fraction one and two were collected for subsequent PhA enrichment (as described in example 1) while fractions 3 to 8 were collected as PhA free EPA/DHA product. FIG. 3c shows the GC chromatogram of such a product containing less than 50 ppm PhA.

### Example 4

A further example for PhA free omega 3 products is shown in example 4. In this case 8 kg of a commercially available 46/08 fish oil ethyl ester was injected onto the fractionation column (aminopropyl-silica column). This oil contains 670 ppm PhA. The separation was done at a temperature of 40° C., a pressure of 140 bar and a CO₂ flow of 700 kg/h.

Table 1 shows the distribution profile of EPA, DHA and PhA in the 8 fractions collected. It can be see that only the first two fractions contain PhA. While fractions 3 to 8 represent different further concentrated PUFA compositions free of PhA.

TABLE 1
PhA distribution in eight SFC fractions (F1.1 to F.4.2) obtained
by injecting a commercially available 46/8 fish oil ethyl
ester concentrate onto a SFC-column.

| | PhA (ppm) | EPA % (m/m) | DHA % (m/m) |
|---|---|---|---|
| Feed (46/08) | 670 | 46 | 8 |
| F.1 | 1700 | 31.5 | 1.5 |
| F.2 | 520 | 47.5 | 1.5 |
| F.3 | <10 | 60.9 | 6.4 |
| F.4 | <10 | 66.5 | 10 |
| F.5 | <10 | 67.0 | 15.1 |
| F.6 | <10 | 48.8 | 28.5 |
| F.7 | <10 | 21.6 | 47.9 |
| F.8 | <10 | 22.7 | 19.5 |

With the above described method it is possible to reduce the phytanic acid (PhA) content in marine oils and other lipid PUFA sources to levels normally found in most of our food (<10 ppm). Hence eliminating the potential toxic risk of PhA and boosting the efficacy of EPA/DHA and other PUFA's in omega-3 food supplements, omega-3 enriched food, omega-3 enriched baby formulas and in omega-3 based cosmetics and pharmaceuticals.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference, further description is deemed unnecessary. In addition, it should be understood that aspects of the invention and portions of various embodiments may be combined or interchanged either in whole or in part. Furthermore, those of ordinary skill in the art will appreciate that the foregoing description is by way of example only, and is not intended to limit the invention.

What is claimed is:

1. A process for fractionation of chromatographic feed material comprising fatty acids and/or derivatives thereof including phytanic acid and/or its derivatives said process comprises subjecting said chromatographic feed material to column chromatography using a liquid or a supercritical fluid as an eluent and collecting a plurality of eluate fractions, one or more of which contain increased amounts of phytanic acid and/or of its derivatives, and one or more of which contain reduced amounts of phytanic acid and/or its derivatives.

2. A process according to claim 1, wherein the fractions containing reduced amounts of phytanic acid and/or its derivative have a content of 0-100 ppm thereof.

3. A process according to claim 1, wherein the fractions containing reduced amounts of phytanic acid and/or its derivative have a content of between 0 and 50 ppm thereof.

4. A process according to claim 1 comprising the steps of:
a) subjecting as a chromatographic feed material an oil comprising more than 100 ppm of phytanic acid and/or of esters or salts thereof and comprising triglycerides containing fatty acid groups to a transesterification reaction to result in a composition comprising alkyl esters of fatty acids,
b) subjecting the composition comprising alkyl esters of fatty acids to column chromatography using a liquid or a supercritical fluid as an eluent,
c) collecting separate fractions from the treatment of step b), and
d) continuing processing of those fractions having a content of phytanic acid and/or of its derivatives of above 100 ppm to result in a product having a total content of phytanic acid and/or of its derivatives of at least triple of the initial quantity found in the chromatographic feed material.

5. A process according to claim 4, wherein the continued processing comprises a combination of those fractions from the column chromatography resulting in a content in the combined fractions of phytanic acid and/or its derivatives of at least than triple of the initial quantity found in the chromatographic feed material.

6. A process according to claim 1 comprising the steps of:
a) subjecting as a chromatographic feed material an oil comprising more than 100 ppm of phytanic acid and/or of esters or salts thereof and comprising triglycerides containing fatty acid groups to a transesterification reaction to result in a composition comprising alkyl esters of fatty acids,
b) subjecting the composition comprising alkyl esters of fatty acids to an urea fractionation process where the saturated and monounsaturated fatty acids and their derivatives are eliminated to a large extent,
c) subjecting the composition from step b) comprising alkyl esters of fatty acids to column chromatography using a liquid or a supercritical fluid as an eluent,
d) collecting separate fractions from the treatment of step c), and
e) continuing processing of those fractions having a content of phytanic acid and/or of its derivatives of above 100 ppm to result in a product having a total content of phytanic acid and/or of its derivatives of at least triple of the initial quantity found in the chromatographic feed material.

7. A process according to claim 6, wherein the continued processing comprises a combination of those fractions from the column chromatography resulting in a content in the combined fractions of phytanic acid and/or its derivatives of at least than triple of the initial quantity found in the chromatographic feed material.

8. A process according to claim 1 comprising the steps of:
a) subjecting as a chromatographic feed material an oil comprising more than 100 ppm of phytanic acid and/or its esters or salts thereof and comprising triglycerides containing fatty acid groups to a transesterification reaction to result in a composition comprising alkyl esters of fatty acids,
b) subjecting the composition comprising alkyl esters of fatty acids to column chromatography using a liquid or a supercritical fluid as an eluent,
c) collecting separate fractions from the treatment of step b), and
d) continuing processing of those fractions having a content of phytanic acid and/or its derivatives of 100 ppm or lower to result in a product having a total content of phytanic acid and/or its derivatives between 0 and 100 ppm.

9. A process according to claim 8, further comprising,
after subjecting the chromatographic feed material to a transesterification reaction, the steps of:
subjecting the composition comprising alkyl esters of fatty acids to an urea fractionation process where the saturated and monounsaturated fatty acids and their derivatives are eliminated to a large extent, and
subjecting the fractionated composition to column chromatography using a liquid or a supercritical fluid as an eluent.

10. A process according to claim 8, wherein the continued processing comprises a combination of those fractions from the column chromatography resulting in a content in the combined fractions of phytanic acid and/or its derivatives of between 0 and 100 ppm.

11. A process according to claim 1, wherein the chromatographic feed material is an oil containing phytanic acid as well as triglycerides containing fatty acid groups.

12. A process according to claim 11, wherein the oil comprises triglycerides containing polyunsaturated fatty acid groups.

13. A process according to claim 11, wherein the chromatographic feed material is selected from the groups of an oil of plant origin, of an oil of marine origin, or of an oil produced by microorganisms, in heterotrophic or autotrophic industrial fermenters, open tubes, flat panel reactors or open pond systems.

14. A process according to claim 1, wherein the eluent is selected from the group of liquid or supercritical dinitrogen oxide, fluorohydrocarbon, fluorochloro-hydrocarbon, carbon dioxide, sulfur hexafluoride, propene, propane, ammonia, sulfur dioxide, xenon, ethane, pentane, hexane, heptane or a mixture of two or more thereof.

15. A process according to claim 1, wherein the liquid or supercritical fluid contains a polar modifier, preferably methanol, ethanol, isopropanol or water.

16. A process according to claim 1, wherein the chromatographic column is packed with silica, or wherein the chromatographic column is packed with aluminium oxide,

17. A process according to claim 16, wherein the silica or aluminium oxide is modified with aminopropyl, with cyanopropyl, with diol, phenyl, or with hydrocarbons.

18. A process according to claim 16, wherein the silica or aluminium oxide is a reverse phase RP 8 or reverse phase RP 18 material.

19. A process according to claim 6, wherein the continued processing comprises a combination of those fractions from the column chromatography having a content of polyunsaturated fatty acid alkyl esters of at least 50% by weight to result in a product having a content of polyunsaturated fatty acid ester of at least 60% by weight and having in total between 0 and 100 ppm of phytanic acid and/or of an alkyl ester and/or of a salt thereof.

20. A process according to claim 19, wherein the continued processing comprises a combination of those fractions from the column chromatography having a content of polyunsaturated fatty acid alkyl esters of at least 50% by weight to result in a product having a total content of EPA alkyl ester and of DHA alkyl ester of at least 80% by weight having in total between 0 and 100 ppm of phytanic acid and/or of an alkyl ester and/or of a salt thereof.

21. A process according to claim 20, wherein the continued processing comprises a combination of those fractions from the column chromatography having a content of polyunsaturated fatty acid alkyl esters of at least 50% by weight to result in a product having a total content of EPA alkyl ester and of DHA alkyl ester of at least 85% by weight and a content of omega-3 alkyl esters of DPA, ETA and 21:5 of below 3% by weight having in total between 0 and 100 ppm of phytanic acid and/or of an alkyl ester and/or of a salt thereof.

22. A process according to claim 21, wherein the continued processing comprises a combination of those fractions from the column chromatography having a content of polyunsaturated fatty acid alkyl esters of at least 50% by weight to result in a product having a total content of 21:5 alkyl ester, fatty acid thereof or salt thereof of less than 1.5% by weight having in total between 0 and 100 ppm of phytanic acid and/or of an alkyl ester and/or of a salt thereof.

23. A composition of matter comprising at least 60% by weight of polyunsaturated fatty acids and/or their derivatives and between 0 and 100 ppm of phytanic acid and/or of its derivatives.

24. A composition of matter according to claim 23, wherein the composition comprises between 0 and 50 ppm of phytanic acid and/or of its derivatives.

25. The composition of claim 23, wherein the polyunsaturated fatty acids and/or their derivatives comprise at least a total of 85% by weight of EPA and DHA and/or of their derivatives and in addition comprise in total between 0 and 3% by weight of omega-3, DPA, ETA and 21:5 and/or their alkyl esters and/or their salts.

26. The composition of claim 25, wherein the polyunsaturated fatty acids and/or their derivatives comprise in total between 0 and 1.5% by weight of 21:5 and/or its alkyl ester and/or its salt.

27. The composition of claim 23, wherein the composition consists essentially of pharmaceutically acceptable constituents.

28. A composition comprising polyunsaturated fatty acids and/or their salts and/or their alkyl esters and comprising between 3000 ppm and 100% by weight of phytanic acid and/or its salts and/or its alkyl esters.

29. A food substance comprising the composition of claim 23.

30. A composition according to claim 28 wherein the composition is a cosmetic composition or as a pharmaceutical composition.
